(19) United States
(12) Patent Application Publication
(10) Pub. No.: US 2011/0196456 A1
(43) Pub. Date: Aug. 11, 2011

(54) SKIN TREATMENT APPARATUS

(75) Inventors: Philip MUTTON, Surrey (GB); Desmond MILLS, Cheltenham (GB)

(73) Assignee: THE DEZAC GROUP LIMITED, Cheltenham (GB)

(21) Appl. No.: 13/021,988

(22) Filed: Feb. 7, 2011

(30) Foreign Application Priority Data
Feb. 5, 2010 (GB) 1001913.1

(51) Int. Cl. A61N 5/06

(52) U.S. Cl. 607/90

(57) ABSTRACT

In an intense pulsed light optical radiation is directed from a lamp 9 to a skin treatment surface to provide cosmetic and/or therapeutic treatment. The condition of the lamp is determined on the basis of a variation of electrical characteristic as it ages, rather then by measuring the light output. The electrical energy applied to the lamp 9 is controlled to compensate for varying electrical characteristics of the bulb as it ages. Furthermore, the device includes an end of life assessment which is based on the electrical characteristic of the bulb 9 rather than simply a cycle count.

This invention relates to apparatus and methods for skin treatment and in particular, but not exclusively, to the treatment of human or animal skin using intense optical radiation pulses to effect a cosmetic and/or therapeutic treatment, for example hair removal. In particular, the invention provides an improved apparatus and method allowing for improved operation of such devices.

It is already known to use intense pulse light (IPL) treatment for hair removal. In this method optical radiation is directed towards the skin with the radiation being absorbed in the hair follicle and on the skin surface. The wavelength of the optical radiation is selected so as to be absorbed by melanin in the follicle so that the hair is heated to a temperature which causes it to inhibit or stop growth. Although incidence of the optical radiation on the skin can also cause local skin heating, the heating of the hair follicle is much more acute.

Traditionally within the art, the optical radiation is generated by a flash bulb such as a gas discharge lamp. Gas discharge lamps are a family of artificial light sources that generate light by sending an electrical discharge through an ionized gas, i.e. a plasma. Typically, such lamps use a noble gas (e.g. argon, neon, krypton or xenon) or a mixture of these gases. The electrodes of the lamp are usually connected to a capacitor, which is charged to a relatively high voltage (generally between 250 and 5000 volts), using a voltage multiplier circuit with e.g. a step up transformer, and a rectifier.

In operation the gas is first ionized, or “triggered”, reducing the electrical resistance of the gas so a spark will form between the electrodes, allowing the capacitor to discharge. The sudden surge of electric current quickly heats the gas to a plasma state, where electrical resistance becomes very low and free electrons, accelerated by the electrical field in the tube, collide with gas and metal atoms. Some electrons circling around the gas and metal atoms are excited by these collisions, bringing them to a higher energy state. When the electron falls back to its original state, it emits a photon, resulting in visible light or ultraviolet radiation.

However, continued use causes the flash bulb to deteriorate and a higher voltage is required to maintain the arc discharge. As a result the capacitor is unable to discharge fully and the optical output of the flash bulb decreases. As a lamp reaches its end of life, the voltage necessary to maintain the arc eventually rises to exceed the voltage provided by the electrical supply and the lamp fails to function. The deterioration of the bulb can be caused by a number of factors including sputtering of the electrodes and ingress of gases between the interior of the flash bulb and the outside environment.

Further, flash lamps operate at high pressures and it is known for some to explode when nearing their end of life, producing violent shockwaves.

Traditionally such lamps are restricted to a predetermined number of uses and are shielded behind glass or in a reflector cavity to mitigate the risk of eye and ear damage as a result of an explosion. One prior art approach to this problem was to provide the device with an electrical counter situated within the bulb arrangement to record the number of uses and inhibit operation when the number of uses reached a predetermined threshold. A common protocol was to limit the bulb use to 2,000 flashes. However, the prior art devices have not fully addressed the bulb deterioration problem and the possibility of explosive failure exists even within the predetermined limit. Further, flash bulbs actually capable of significantly higher number of safe cycles are thereby prematurely removed from use resulting in an expensive and unnecessary maintenance regime. Also, the gradual deterioration of the bulb means that the amount of energy emitted for discharge is not uniform throughout the bulb life.

Accordingly there is a need for a skin treatment apparatus with an optical radiation source, which includes means for monitoring the condition of the source. Also there is a need for a skin treatment apparatus that allows for a prolonged operational optical output of the source and where the operational life of the flash bulb may be assessed other than simply on the basis of the number of flashes. A desirable feature is for the apparatus to contain means for monitoring the source and inhibiting the operation of the source when signs of approaching failure occur. Likewise, similar considerations apply to light treatment devices other than Intense Pulse Light devices. Furthermore it is desirable to provide a method for assessing bulb life and condition which does not depend on measuring light output directly.

### SUMMARY OF THE INVENTION

Accordingly, in one aspect, this invention provides skin treatment apparatus for the treatment of human or animal skin by optical radiation, said apparatus including:

a source of optical radiation for directing optical radiation towards a treatment area of the skin;

electrical storage means for providing energy for said optical radiation source, and

a source condition detector for monitoring at least one electrical parameter of said source and/or said electrical storage means, thereby to derive data indicative of the condition of said optical radiation source.

Preferably said optical radiation source comprises an electrically operated gas discharge lamp, and said source condition detector is operable to monitor at least one discharge electrical parameter of the discharge lamp during or after, the discharge. Conveniently, the measure is made at or shortly after completion of the discharge. Alternatively, as the discharge is exponential it is possible to measure the electrical parameter part-way through the discharge and to detect the condition, knowing the discharge profile.

Conveniently at least one of said discharge electrical parameters is a voltage across said discharge lamp. This may be measured directly or by measuring the voltage across the energy storage means.

The apparatus may generate a pulse which extinguishes when the voltage across the lamp is no longer of sufficient magnitude to maintain the discharge, or it may include timer means for inhibiting discharge after a preset period.

In one arrangement said source condition monitor is operable to detect whether said discharge voltage is below a preset threshold within a preset period following initiation of the discharge.

In another arrangement said source condition detector monitors a voltage across said discharge lamp immediately before said discharge and a voltage during or after the discharge. Again the voltage immediately before the discharge may be measured directly or by measuring the voltage across the energy storage means.

In this arrangement said source condition detector preferably monitors the difference between the respective voltages immediately before and during or after the discharge.

Although the apparatus may simply use the derived data as an indicator of the present condition of the discharge lamp it may also be used to indicate end of life. For example, the apparatus could monitor the voltage taken during or after the discharge to indicate an end of life condition when said voltage exceeded a preset value. Furthermore it may be advantageous to use such data to control the electrical energy supplied to the discharge lamp to compensate for ageing of the lamp.

Thus, the skin treatment apparatus advantageously includes energy control means responsive to said source condition detector to adjust the energy delivered to said optical radiation source. The energy control means may for example control at least one of the magnitude and duration of the voltage pulse applied to said optical radiation source.

In one particular arrangement said energy control means controls both the magnitude and duration.

In one embodiment the source condition detector determines the difference between the respective voltages across the discharge lamp before and during or after a discharge and compares said difference with a preset value, and correspondingly increases at least one of the magnitude of the initial voltage, and the duration of the applied voltage for subsequent operation in accordance with the results of said comparison.

In a preferred arrangement said energy control means determines a correction factor by comparing a first voltage difference between the respective voltages across the discharge lamp before and during or after a pre-selected discharge and a second voltage difference across the discharge lamp before and after a later given discharge, and thereafter adjusts the initial voltage for subsequent discharges by an amount dependent on said correction factor.

This may be done incrementally by comparison with the relevant parameters for a previous time when the correction factor was adjusted, or the full correction factor may be calculated each time using baseline figures from a calibration routine at the start of the life of the discharge lamp.

In one particular arrangement, the said correction factor (CF) is determined using the following formula:

CF=K·SQRT[(V1−V2)²−(V3−V4)²], where

V1=initial voltage at previous calibration

V2=end voltage at previous calibration

V3=initial voltage at present re-calibration

V4=end voltage at present re-calibration

K is a constant, which may be unity.

Furthermore, the apparatus may modulate the correction factor in accordance with a temperature measurement, so

CF=K·T·SQRT[(V1−V2)²−(V3−V4)²]

where T=temperature.

Thus, in either case, having determined the correction factor, the initial voltage applied across the discharge lamp is incremented by CF volts. The treatment apparatus may conveniently include an end of life detector responsive to variations in the voltage applied to said discharge lamp by said energy correction means to provide an end of life indication. Said end of life detector may indicate an end of life condition when the accumulated increase applied to the initial voltage exceeds a preset amount. Likewise, said end of life detector may monitor said correction factor and provides an end of life condition indication when the correction factor exceeds a preset amount.

Instead of adjusting the trigger or initial voltage, said energy control means may be operable to adjust at least one of the duration and initial magnitude of an electrical pulse adapted to energise said optical radiation source.

In another arrangement said optical radiation source may be energised by a series of electrical pulses, and said energy control means may operable to adjust at least one of the number of pulses, the pulse width duration, the inter-pulse delay and pulse amplitude of the series of pulses.

Preferred embodiments of this invention provide a hair treatment device for the treatment of the human or animal skin by optical radiation to prevent or reduce hair growth, which device comprises:

a source of optical radiation for emitting an intense pulse of optical radiation;

an energy storage means connected to said source;

an energy delivery means for discharging said energy storage into said source;

a monitoring means for monitoring the electrical parameters of said source and storage capacitor, and

a control means arranged to receive signals from said monitoring means operable to control said energy delivery means.

The treatment effected thereby may be exclusively cosmetic treatment, exclusively therapeutic treatment or a mixture thereof. For example, the treatment may comprise one or more of:

removal of hair,

removal of tattoos or other skin pigmentation,

treatment of visible capillaries such as port wine stains or surface veins, rosacea and similar disorders,

treatment to reduce the appearance of cellulite.

Preferably, said optical radiation source comprises a flash bulb source. The flash bulb source may take many forms, but in one arrangement may be a gas discharge lamp or flash bulb.

Said energy storage means may take many forms but preferably comprises a storage capacitor. Other storage means could be used to supply sufficient energy to said optical radiation source including for example high output batteries.

The energy delivery means may take many different forms but is preferably a microprocessor connected to a trigger circuit via an opto-isolator. Said trigger circuit may comprise a Thyristor electrically coupled to a high voltage trigger coil and an insulated gate bipolar transistor (IGBT) semi conductor switch electrically coupled in series between said energy storage means and optical source operable to control the output of said energy storage means to said optical source.

The monitoring means may monitor a range of electrical parameters of said optical source or energy storage means, but preferably monitors the voltage present on said energy storage means. In one arrangement, an opto-isolator is used to measure the voltage present on the storage capacitor terminals during electrical charge and discharge and transmit this measurement as an electrical signal.

The control means may take many different forms but is preferably a microprocessor capable of receiving signals from said monitoring means, for comparing said signal to a predetermined threshold and operable to control said energy delivery means. In one arrangement, the control means further includes a timer and memory.

Accordingly in operation of preferred embodiments, electrical parameters of said energy storage means and flash bulb are measured by the monitoring means and altered by said control means to compensate for flash bulb impedance differences by changing the nature of the delivered electrical pulse from said energy storage means to said flash bulb.

In some embodiments, the electrical pulse duration and amplitude are predetermined values.

Accordingly in other embodiments, the duration and amplitude of the electrical pulse may be extended if the amplitude of a previous electrical discharge pulse fails to fall to a preset threshold value by the end of the predetermined duration, as might occur with a worn high impedance bulb.

In further embodiments, the duration and amplitude of the electrical pulse may be extended if the amplitude of said electrical discharge test pulse fails to fall to a preset threshold value by the end of the predetermined duration, as might occur with a worn high impedance bulb.

In yet other embodiments, multiple electrical pulses are discharged from said energy storage means to said flash bulb. The number of pulses, pulse width duration, inter pulse delay and pulse amplitude of the electrical pulses may be altered if the amplitude of an electrical pulse fails to fall to a threshold value by the end of the predetermined duration, as might occur with a worn high impedance bulb.

This method and apparatus of altering the delivered electrical pulse thereby compensates for impedance differences in the flash bulb by changing the nature of the delivered electrical pulse or pulses to the flash bulb, resulting in a smaller, more efficient and less expensive device.

Whilst the invention has been described above, it extends to any inventive combination or sub-combination of the features as set out above, or in the following description or drawings.

The invention may be performed in various ways, and an embodiment thereof will now be described by way of example only, reference being made to the accompanying drawings, in which:

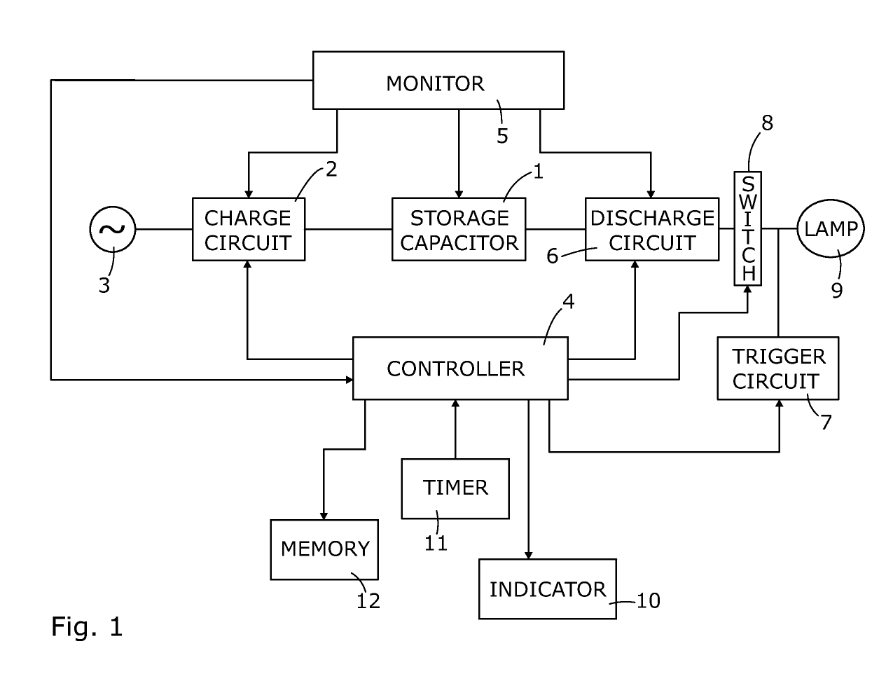

FIG. 1 is a schematic view of an embodiment of the present invention;

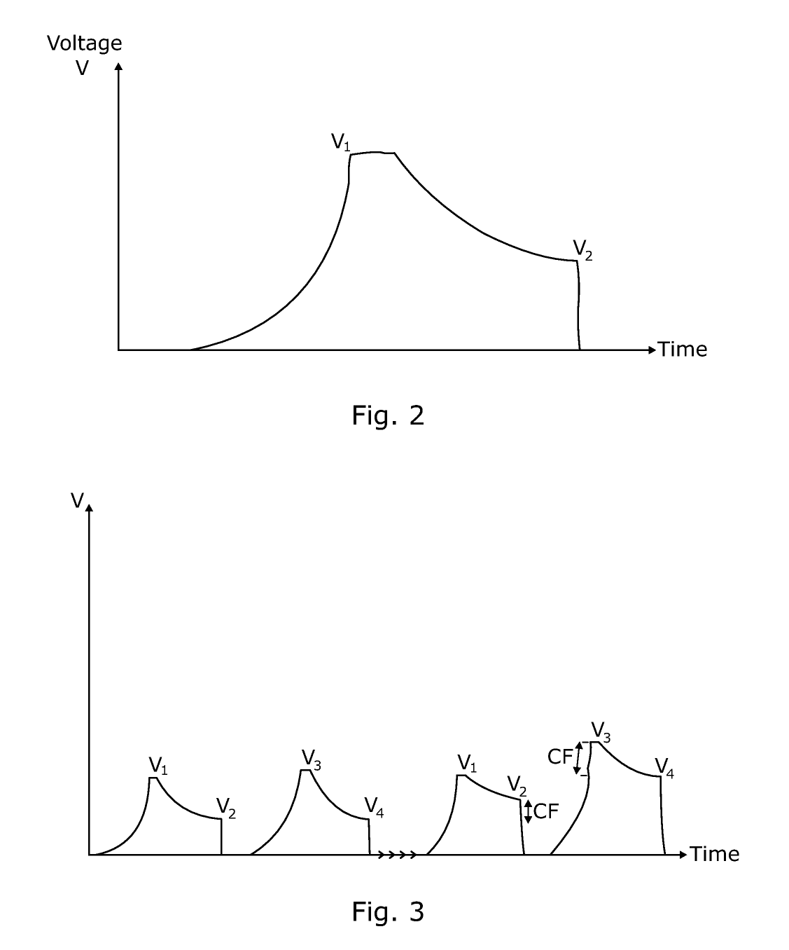

FIG. 2 is a pulse diagram showing a calibration pulse;

FIG. 3 is a pulse diagram showing an ongoing calibration for multiple pulses;

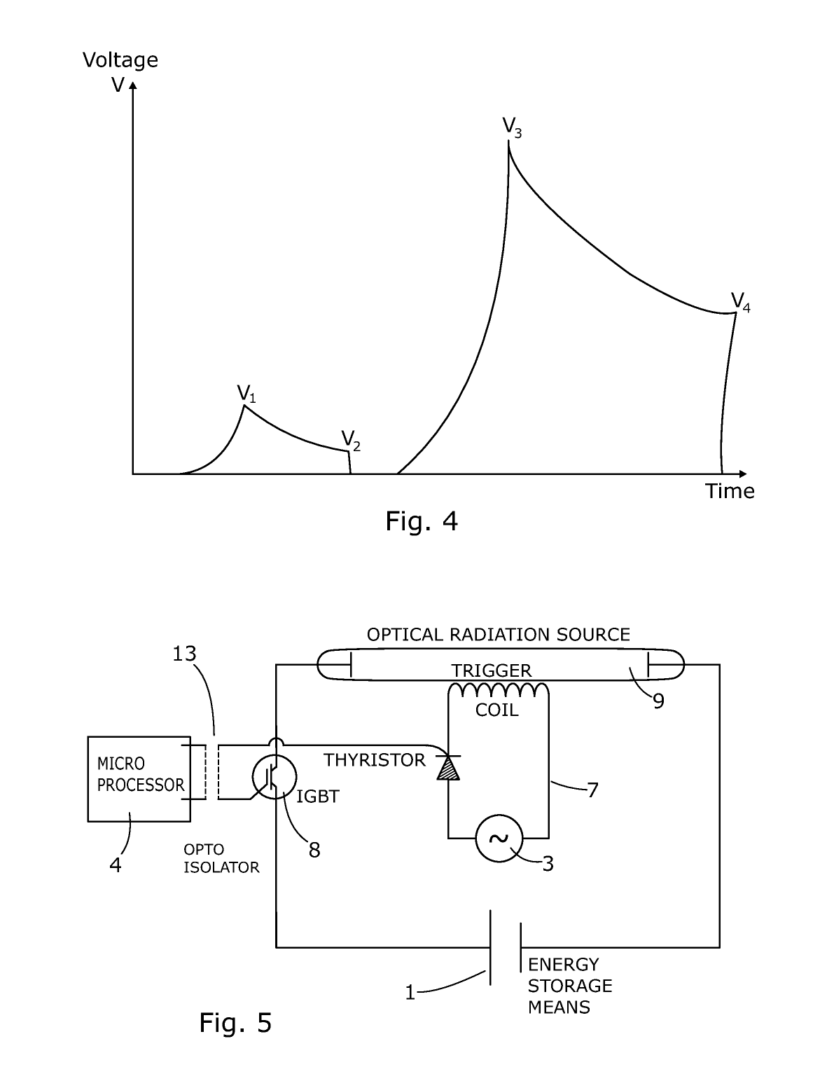

FIG. 4 is a pulse diagram showing a test pulse prior the treatment pulse, and

FIG. 5 is a circuit diagram of the discharge lamp, the trigger coil, and associated circuitry.

Referring initially to FIG. 1, there is shown a schematic diagram of an IPL device constructed in accordance with this invention. In this device, optical radiation is directed from a lamp 9 to a skin treatment surface to provide cosmetic and/or therapeutic treatment. The electrical energy applied to the lamp 9 is controlled to compensate for varying electrical characteristics of the bulb as it ages. Furthermore, the device includes an end of life assessment which is based on the electrical characteristic of the bulb 9 rather than simply a cycle count.

In the device electrical power from a mains source 3 passes via a charge circuit 2, which raises the voltage by a transformer and rectifies it, to a storage capacitor 1. Once the storage capacitor has been charged to a preset level the device is ready to be fired. The fire signal is initiated by a push button or the like which activates a trigger circuit 7. Upon activation of the trigger circuit 7, a discharge circuit 6 applies the voltage in the storage capacitor 1 via a switch 8 to the lamp 9 thereby generating a pulse of optical light. Operation and control of the device are effected by means of a controller 4 which controls the charge circuit, the storage capacitor 1, discharge circuit 6 and semi conductor switch 8. A monitor 5 monitors signal levels on the charge circuit 2, the storage capacitor 1 and the discharge circuit 6 and supplies data to the controller 4. The controller 4 includes a timer 11, a memory 12 and a warning indicator 10.

In use a storage capacitor 1 is charged by charge circuit 2 from mains supply 3 under the control of a controller 4. The charge voltage of storage capacitor 1 V1 is monitored by a monitor means 5 and a signal representative of the voltage SV1 is transmitted by the monitor means 5 to controller 4. When the signal SV1 reaches a predetermined threshold (PDT), control means (5) deactivates charge circuit (2).

The controller 4 operates the discharge circuit 6 triggering trigger circuit 7 and closing IGBT 8 so that the storage capacitor 1 discharges into the discharge bulb 9 causing a spontaneous flash of optical energy to be emitted from the device. As will be apparent from FIG. 5, the trigger circuit effectively conditions the bulbs so that a discharge is possible between the high voltage contacts. It consists of a coiled wire around the bulb, which when energised (by the trigger circuit) ionises the gas within the bulb and resultantly reduces its resistance. The IGBT 8 and discharge circuit 6 are in a separate circuit connected to the high voltage discharge contacts of the bulb. When the IGBT 8 closes, the energy from the storage capacitor is presented across the bulbs high voltage contacts; as the gas has been previously ionised by the trigger circuit, a discharge is possible—and the flash occurs. The IGBT 8 and a thyristor in the trigger circuit are controlled by the controller 4 via an opto-isolator 13.

Depending on the operating mode, delivery of the electrical pulse may be terminated by a timer 11 after the end of a predetermined period or when the voltage across the electrodes has dropped below a predetermined value as measured by the monitor means 5. The controller 4 terminates the electrical pulse by deactivating discharge circuit 6 and closing IGBT 8 preventing further discharge from storage capacitor 1 into the flash bulb 9.

The controller 4 records the final voltage V2 remaining on the storage capacitor 1 via signal SV2 transmitted from the monitor 5.

The controller 4 calculates the energy transferred from the storage capacitor 1 to the flash bulb 9 from the equation

Eo=k(V1−V2)

where;

Eo=optimal energy transferred from storage capacitor 1 to the flash bulb 9,

k=capacitive constant,

V1=initial storage capacitor voltage

V2=final storage capacitor voltage

The calibrated optimal energy value Eo is stored in memory 12.

Over an extended number of uses flash bulb 9 will wear, the resistance of flash bulb 9 will increase and the value (V1−V2) will decrease. Thus, the energy En transferred from storage capacitor 1 to the flash bulb 9 for a particular electrical discharge n will differ from the stored value Eo.

To accommodate the loss of energy (Eo−En) the controller 4 increases PDT by a corrective factor CF so as to compensate.

The increase in PDT is given by the equation;

PDT_{new}=PDTₚᵣₑᵥᵢₒᵤₛ+CF

Where;

CF=SQRT[(V1−V2)²−(V3−V4)²]

Where;

PDT_{new}=new predetermined threshold

PDTₚᵣₑᵥᵢₒᵤₛ=previous predetermined threshold

CF=Corrective factor

V1=calibrated optimal initial storage capacitor voltage

V2=calibrated optimal end storage capacitor voltage

V3=previous pulse initial storage capacitor voltage

V4=previous pulse storage capacitor end voltage

In a further embodiment calibration is performed by a short low energy calibration pulse triggered prior to each treatment pulse as shown in FIG. 4. Discharge may be initiated manually by the user or automatically in response to a signal from control means 4.

In a further embodiment calibration is performed once following the insertion of flash bulb 9 and predetermined values of CF are used to provide optimal pulse efficacy.

For end of life detection, the monitor 5 monitor the CF and where this CF is greater than a predetermined end of life threshold (EOL) control means 4 inhibits operation of the flash bulb 9 and sends a warning signal to warning indicator 10.

1. Skin treatment apparatus for the treatment of human or animal skin by optical radiation, said apparatus including:
a source of optical radiation for directing optical radiation towards a treatment area of the skin;
electrical storage means for providing energy for said optical radiation source, and
a source condition detector for monitoring at least one electrical parameter of said source and/or said electrical storage means, thereby to derive data indicative of the condition of said optical radiation source.

2. Skin treatment apparatus according to claim 1, wherein said optical radiation source comprises an electrically operated gas discharge lamp, and said source condition detector is operable to monitor at least one discharge electrical parameter of the discharge lamp at, or shortly after, completion of the discharge.

3. Skin treatment apparatus according to claim 2, wherein at least one of said post discharge electrical parameters is a voltage across said discharge lamp.

4. Skin treatment apparatus according to claim 2, including timer means for inhibiting discharge after a preset period.

5. Skin treatment apparatus according to claim 3, wherein said source condition monitor is operable to detect whether said post discharge voltage is below a preset threshold within a preset period following initiation of the discharge.

6. Skin treatment apparatus according to claim 3, wherein said source condition detector monitors respective voltages across said discharge lamp immediately before said discharge and at, or shortly after, completion of the discharge.

7. Skin treatment apparatus according to claim 6, wherein said source condition detector monitors the difference between the respective voltages immediately before and at, or shortly after, completion of the discharge.

8. Skin treatment apparatus according to claim 1, including energy control means responsive to said source condition detector to adjust the energy delivered to said optical radiation source.

9. Skin treatment apparatus according to claim 8, wherein said energy control means is operable to control at least one of the magnitude and duration of the voltage pulse applied to said optical radiation source.

10. Skin treatment apparatus according to claim 9, wherein said energy control means controls both the magnitude and duration.

11. Skin treatment apparatus according to claim 9, wherein said source condition detector is operable to determine the difference between the voltage across the discharge lamp before and after a discharge and to compare said difference with a preset value, and to correspondingly increase at least one of the initial voltage and the duration of the applied voltage for subsequent operation in accordance with the results of said comparison.

12. Skin treatment apparatus according to claim 11, wherein said energy control means determines a correction factor by comparing a first voltage difference between the respective voltages across the discharge lamp before and after a pre-selected discharge and a second voltage difference across the discharge lamp before and after a later given discharge, and adjusting the initial voltage for subsequent discharges by an amount equal to said correction factor.

13. Skin treatment apparatus according to claim 12, including means for updating said correction factor incrementally based on changes in said voltage differences since the last calibration routine.

14. Skin treatment apparatus according to claim 12, wherein said correction factor (CF) is determined using the following formula:
CF=K·SQRT[(V1−V2)²−(V3−V4)²], where
V1=initial voltage at previous calibration
V2=end voltage at previous calibration
V3=initial voltage at present re-calibration
V4=end voltage at present re-calibration
K is a constant.

15. Skin treatment apparatus according to claim 8, including an end of life detector responsive to variations in the voltage applied to said discharge lamp by said energy control means to provide an end of life indication.

16. Skin treatment apparatus according to claim 13, wherein said end of life detector indicates an end of life condition when the accumulated increase applied to the initial voltage exceeds a preset amount.

17. Skin treatment apparatus according to claim 12,
further comprising means for updating said correction factor incrementally based on changes in said voltage differences since the last calibration routine;
wherein said end of life detector indicates an end of life condition when the accumulated increase applied to the initial voltage exceeds a preset amount;
wherein said end of life detector monitors said correction factor and provides an end of life condition indication when the correction factor exceeds a preset amount.

18. Skin treatment apparatus according to claim 8, wherein said energy control means is operable to adjust at least one of the duration and initial magnitude of an electrical pulse adapted to energise said optical radiation source.

19. Skin treatment apparatus according to claim 8, wherein said optical radiation source is energised by a series of electrical pulses and said energy control means is operable to adjust at least one of the number of pulses, the pulse width duration, the inter-pulse delay and pulse amplitude of the series of pulses.

20. Skin treatment apparatus according to claim 1, including means for inhibiting operation of the optical radiation source upon detection of an end of life condition.

21. Skin treatment apparatus according claim 1, including means for providing an audible or visual indication of the condition of the optical radiation source.
